(19) United States
(12) Patent Application Publication
(10) Pub. No.: US 2012/0215074 A1
(43) Pub. Date: Aug. 23, 2012

(54) ENDOTRACHEAL TUBE WITH SENSORS

(76) Inventor: William Krimsky, Bel Air, MD (US)

(21) Appl. No.: 13/257,840

(22) PCT Filed: Mar. 22, 2010
(86) PCT No.: PCT/US10/28127

§ 371 Date: May 7, 2012

(30) Foreign Application Priority Data
Mar. 20, 2009 (US) 61161785

(51) Int. Cl. A61M 16/04; A61B 8/12; A61B 5/0205; A61B 18/02; A61B 5/029; A61B 5/00; A61B 8/06

(52) U.S. Cl. 600/301; 600/300; 600/462; 600/465; 600/526

(57) ABSTRACT

The present invention provides an endotracheal tube comprising one or more sensors. n some embodiments, one or more sensors are positioned in the interior of the cuff. Sensors ay be used to detect any physiological parameter.

### CROSS-REFERENCE TO RELATED APPLICATIONS

This application claims priority to U.S. Provisional Application for Patent Ser. No. 61/161,785 filed Mar. 20, 2009, the entire contents of which are specifically incorporated herein by reference.

### BACKGROUND OF THE INVENTION

An endotracheal tube (also called an ET tube or ETT) is used in general anesthesia, intensive care and emergency medicine for airway management and mechanical ventilation. The tube is inserted into a patient's trachea in order to ensure that the airway is not closed off and that air is able to reach the lungs.

There are many types of endotracheal tubes which range in size from 2-10.5 mm in internal diameter (ID). The proper size endotracheal tube is selected based on the patient's body size with the smaller sizes being used for pediatric and neonatal patients.

Endotracheal tubes are typically equipped with an inflatable cuff at or near their distal end. After insertion of the endotracheal tube, the cuff may be inflated to form a seal between the outer surface of the endotracheal tube and the trachea. This helps to create a closed system to allow for the typical increase driving pressures used in mechanical ventilation as well as to reduce movement of the endotracheal tube ensuring that it remains where positioned. When properly positioned, the distal end of an endotracheal tube is typically located in the trachea in proximity to the ascending aorta, which is an optimum position from which to measure a variety of physiological parameters including cardiac output.

Cardiac output is the volume of blood which the heart pumps in one minute and is one of the most important cardiovascular parameters. The cardiac output reflects the supply of oxygen and nutrients to tissue. Measurements of cardiac output provide invaluable clinical information for quantifying the extent of cardiac and vascular dysfunction, indicating the optimal course of therapy, managing patient progress, and establishing check points for rehabilitation in a patient with a damaged or diseased heart, or one in whom fluid status control is essential.

Cardiac output can be measured using ultrasound and the Doppler Effect. The blood velocity through the heart causes a ‘Doppler shift’ in the frequency of the returning ultrasound waves. This Doppler shift can then be used to calculate flow velocity and volume and subsequently cardiac output. Doppler ultrasound is non-invasive, accurate and inexpensive and is a routine part of clinical ultrasound with high levels of reliability and reproducibility having been in clinical use since the 1960s.

Ultrasound is performed by emitting a pulse of high frequency sound (typically in the 1-12 MHz range) into a target and then measuring the reflected sound waves. Since ultrasound does not propagate well in gas, it is important that the ultrasound generator (e.g., a piezoelectric crystal) be in intimate contact with the target material.

Endotracheal tubes comprising ultrasound transducers are described in U.S. Pat. Nos. 4,671,295 to Abrams et al., 4,722,347 to Abrams et al. and 5,076,268 to Weber. The Abrams et al. patents address the requirement for intimate contact between ultrasound transducer and target by mounting one or more transducers on the tube and equipping the tube with variously shaped inflatable balloons to urge the ultrasound transducers into contact with the inner wall of the trachea. U.S. Pat. No. 5,076,268 issued to Weber discloses an endotracheal tube having an ultrasound transducer assembly mounted at one end of the tube and an asymmetrically disposed balloon cuff to simultaneously seal the trachea and urge an ultrasound transducer into contact with the inner wall of the trachea. As acknowledged by Abrams et al., the inner wall of the trachea is irregularly shaped and relatively non-deformable cartilaginous tissue. To ensure a good contact between the ultrasound transducer and the inner wall of the trachea, the inventors suggest the use of an acoustical gel. Notwithstanding these arrangements, continuous contact between the tracheal wall and the ultrasound device is not adequately maintained by these devices.

There remains a need in the art for an endotracheal tube capable of monitoring physiological parameters including cardiac output. Such an endotracheal tube may be adapted to provide an acoustically transmissive contact between one or more ultrasound transducers and one or more targets. This need and others are met by the present invention.

### SUMMARY OF THE INVENTION

The present invention provides materials and methods for monitoring physiological parameters including cardiac output. In some embodiments, the present invention provides an endotracheal tube. Such endotracheal tubes may comprise an elongate tubular member having a proximal end and a distal end and one or more flexible cuffs. Flexible cuffs of the invention may be positioned on the exterior of the elongate tubular member. A flexible cuff of the invention may have an interior space and one or more transducers (e.g., ultrasound transducers, sensors, transmitters etc) positioned in the interior space of the cuff. In some embodiments, 2, 3, 4, 5 or more transducers (e.g., ultrasound transducers, sensors, transmitters etc), which may be adapted to monitor the same or different physiological parameters, may be positioned in the interior of the cuff. In addition, one or more (e.g., 2, 3, 4, 5 etc) transducers (e.g., ultrasound transducers, sensors, transmitters etc), may be positioned on and/or in the tubular member not within the interior of the cuff. Typically, one or more transducers (e.g., ultrasound transducers, sensors, transmitters etc) positioned in the interior of the cuff comprises an ultrasound transducer, for example, an ultrasound transducer adapted to detect blood flow and/or measure cardiac output and/or monitor other hemodynamic parameters (e.g., left atrial occlusion pressure, vascular resistance, etc.).

In some embodiments, a flexible cuff of the invention may be inflated with an acoustically transmissive material. As used herein, an acoustically transmissive material is one that transmits ultrasound. Suitable examples of acoustically transmissive materials include, but are not limited to, fluids (e.g., water, saline solution, buffer solutions etc) and gels (e.g., acoustical gels). The flexible cuffs of the invention are typically constructed of a material that is sufficiently flexible to allow a contact with the inner wall of the trachea that allows ultrasound waves produced by a transducer in the interior of the cuff to pass into and through the inner wall of the trachea. Optionally, the exterior of the flexible cuff may be coated with an acoustically transmissive material such as an acoustical gel. In one particular embodiment, the invention comprises an endotracheal tube comprising an elongate tubular member and a flexible cuff disposed at or near the distal end of the member wherein an ultrasound transducer is positioned within the interior of the cuff and the cuff is filled with an acoustically transmissive material (e.g., a fluid or gel).

The present invention also provides methods of performing surgical procedures. Such methods may comprise inserting into a patient an endotracheal tube as described above, using at least one transducer to detect at least one parameter (for example, cardiac output); and performing the procedure. Any surgical procedure known in the art may be performed. Surgical procedures may comprise applying a cryogen to a target tissue or using the cryogen to create an isotherm in proximity to the tissue. In methods of this type, cryogen may applied to a tissue selected from the group consisting of esophageal tissue, lung tissue, tracheal tissue, laryngeal tissue, pharyngeal tissue and gastric tissue. Any suitable cryogen may be used, for example, the cryogen may be a liquefied gas. Examples of suitable cryogens include, but are not limited to, nitrogen, oxygen, argon, carbon dioxide, nitrogen dioxide, nitrogen oxides, and air. Any suitable technique may be used to apply the cryogen, for example, the cryogen can be sprayed (e.g., from a catheter).

### BRIEF DESCRIPTION OF THE DRAWINGS

FIG. 1 is a schematic representation of an endotracheal tube of the invention.

FIG. 2 is a schematic representation of an adjustably mounted transducer.

### DETAILED DESCRIPTION OF THE INVENTION

Endotracheal tubes are currently used as a means to provide ventilator support in a variety of medical situations, for example, during surgery. The present invention provides endotracheal tubes that can be equipped with a variety of different transducers (e.g., ultrasound transducers, sensors, transmitters etc). Suitable transducers include, but are not limited to, ultrasound transducers, temperature sensors, blood gas sensors (e.g., CO₂ and/or O₂ sensors, and infrared sensors to measure tissue oximetry), air flow sensors, sensors for an esophageal electrocardiogram, one or more sensors for sampling of exhaled gases, for example, nitrous or nitric oxide, and sensors for measuring humidity. Any sensor known to those skilled in the art that provides information of interest to an attending physician can be used in the practice of the invention. Information from the sensors can be used to measure metabolic rates, for example, the % consumption of oxygen and % production of CO₂ provide a measure of metabolism which can be used to monitor patient status in critical care situations.

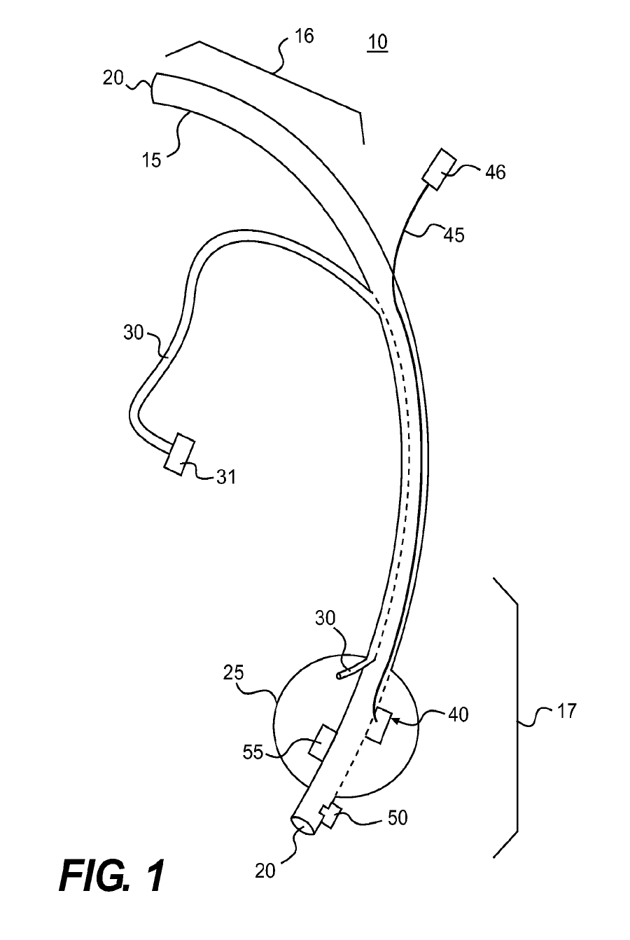

FIG. 1 provides an endotracheal tube 10 of the invention. The endotracheal tube 10 includes elongate tubular member 15 that defines a passage 20 that allows fluid communication between the ambient atmosphere and the patient's lungs.

Endotracheal tube 10 also has a flexible cuff 25 that can be inflated to form a seal between the outer surface of the endotracheal tube and the inner wall of the trachea. The endotracheal tube 10 also includes an inflation tube 30 in fluid communication with the interior of cuff 25 through which material may be introduced into and may be removed from the interior of the cuff. Typically, such material will be acoustically transmissive. The distal end of tube 30 may be equipped with a valve 35 which can be used to control the flow of material into and out of the cuff.

Endotracheal tube 10 also includes a transducer (e.g., ultrasound transducer, sensor, transmitter etc) 40 positioned in the interior of cuff 25. Transducer 40 may be an ultrasound transducer. Transducer 40 may be electrically coupled to any suitable device, for example, a directional pulsed or continuous wave Doppler ultrasound device, via electrical conductor 45 which may be attached to a connector 46. As shown, transducers (e.g., ultrasound transducers, sensors, transmitters etc) of the invention are electrically coupled through wires to suitable devices for recording their output and/or directing input to them. Those skilled in the art will appreciate that transducers equipped with wireless transmission capability may be used and signals to and from such transducers may be sent and/or received by any suitable device.

In one embodiment, transducer 40 may emit an ultrasonic wave through the wall of the trachea and through the wall of the ascending aorta or the pulmonary artery and be reflected by the blood in the selected artery. Due to the movement of the blood in the artery, the reflected signal will have a different frequency than the transmitted signal and this difference (the Doppler shift) can be used to calculate the flow. The ultrasound waves are also reflected by the walls of the artery and these reflected waves can be used to calculate the cross sectional area of the artery.

As shown in FIG. 1, endotracheal tube 10 is equipped with additional transducers (e.g., ultrasound transducers, sensors, transmitters etc) 50 and 55. In some embodiments, endotracheal tube 10 may be equipped with a plurality of transducers, for example, 2, 3, 4, 5, 6, 7, 8, 9, 10 or more. Additional transducers (e.g., sensors) may be used to detect any desired physiological parameter. Suitable sensors include, but are not limited to, temperature sensors, blood gas sensors (e.g., CO₂ and/or O₂ sensors, and infrared sensors to measure tissue oximetry), air flow sensors, sensors for an esophageal electrocardiogram, sensors for sampling of exhaled gases, for example, nitrous or nitric oxide, and sensors for measuring humidity. Any sensor known to those skilled in the art that provides information of interest to an attending physician can be used in the practice of the invention. Information from the sensors can be used to measure metabolic rates, for example, the % consumption of oxygen and % production of CO₂ provide a measure of metabolism which can be used to monitor patient status in critical care situations.

A transducer may or may not be equipped with an electrical conductor. In some embodiments, each transducer may have its own electrical conductor. Typically, an electrical conductor 45 will convey an electrical signal to and from a transducer to an additional piece of hardware appropriate for controlling the transducer and detecting the physiological parameter to be determined by the specific transducer. Electrical conductor 45 may be attached to the hardware via connector 46. Any suitable type of connector may be used in the practice of the invention for example, a plug. For example, when the transducer is a piezoelectric ultrasound transducer, the electrical conductor may convey an alternating current to the transducer to induce production of the ultrasound wave and/or may convey the reflected signal back to a conventional ultrasound machine Suitable ultrasound machines are known to those skilled in the art. Suppliers of suitable ultrasound devices include, but are not limited to, Siemens Medical, Edwards Life Sciences, Phillips Healthcare, and Olympus. Those skilled in the art will appreciate that an electrical conductor may comprise more than one single conductor. In some embodiments, an electrical conductor may be a plurality of wires. In some embodiments, a transducer may not be equipped with an electrical conductor, in such cases, the transducer may receive and/or transmit a signal via radio wave or other form of electromagnetic radiation. Electrical conductors may be embedded in elongate tube 15, run inside of passage 20, run along the outside of elongate tube 15, or be affixed to elongate tube 15 either on the inside or outside. In embodiments comprising a plurality of transducers equipped with electrical conductors, each electrical conductor may independently be arrange in one of these fashions.

Transducers (e.g., ultrasound transducers, sensors, transmitters etc) may be attached to the endotracheal tube of the invention by any method or material known to those in the art. For example, a transducer may be permanently affixed to the endotracheal tube by heat sealing or adhesive. A transducer may be removeably attached to the endotracheal tube of the invention, for example, by friction fitting the transducer in an appropriately shaped receptacle formed on the endotracheal tube. In some embodiments, the endotracheal tube of the invention may be equipped with a socket to which one or more electrical conductors is attached and a transducer may be equipped with one or more prongs adapted to engage holes in the socket to affix the sensor.

One or more transducers (e.g., ultrasound transducers, sensors, transmitters etc) may be in fluid communication with the passage 20 so as to sample the gas moving through the passage. Sampling the gas in the passage may be used to detect the concentration of one or more additives to the gases moving through the passage, for example, an anesthetic and a sensor may be used to monitor the concentration of anesthetic being administered to a patient. Any other characteristic of the gas may be measured, for example, CO₂ and/or O₂ content, rate of gas flow, content and/or concentration of exhaled gases, for example, nitrous or nitric oxide, and humidity. One or more of transducers 50 and 55 may be used to sense the temperature of the patient. In some embodiments, a sensor for determining the temperature of a patient may comprise a thermocouple.

In FIG. 1, the elongate tubular member 15 is depicted as a flexible curved tube. The endotracheal tubes of the invention may take any shape and size known to those skilled in the art and may flexible or rigid. An elongate tubular member 15 of the invention may be constructed of any suitable material used for endotracheal tubes known in the art, e.g., any biocompatible material. Suitable examples of material that may be used to construct the endotracheal tubes of the invention include, but are not limited to, polyvinylchloride, latex, silicone, rubber, or other materials readily apparent to those skilled in the art. In one embodiment, an endotracheal tube of the invention may be constructed of a clear biocompatible material.

As shown in FIG. 1, the elongate tubular member 15 is curved and such a device may be inserted into the trachea via the mouth. Other shapes may be used for insertion through the nose and/or a tracheotomy.

The elongate tubular member 15 has a proximal portion 16 and a distal portion 17. As used herein, proximal is used to indicate the portion of the endotracheal tube that lies outside of the patient after installation and distal is used to indicate the portion of the endotracheal tube that lies inside of the trachea of the patient after installation. Typically, the proximal portion 16 exits either the nasal cavity or the mouth of the patient when installed in a patient In some embodiments, the proximal portion may exit through a tracheostomy. When installed, passage 20 provides fluid communication from the outside of the patient to the lungs. As will be readily appreciated by those in the art, the proximal portion of the endotracheal tube of the invention may be attached to any device typically used to introduce gas into the lungs of the patient, for example, a bag ventilator or a mechanical ventilator.

The endotracheal tube 10 also comprises one or more cuffs 25. The endotracheal tube shown in FIG. 1 comprises a single cuff; however, those skilled in the art are aware that endotracheal tubes comprising multiple cuffs may be constructed and such tubes are within the contemplated scope of the invention. A cuff of the invention is typically constructed of a flexible, biocompatible material, e.g., a biocompatible plastic such as polyvinyl chloride. In FIG. 1, cuff 25 is shown inflated. Any suitable material may be used to inflate cuff 25. A material is suitable if it transmits ultrasound waves. Examples, include, but are not limited to, fluids (e.g., water, saline solution, buffer solutions and the like) and gels (e.g., acoustical gels). When inflated, cuff 25 forms a seal against the inner wall of the trachea that permits ultrasound waves to be transmitted from transducer 40 through the cuff 25, through the wall of the trachea and into the patient. As depicted in FIG. 1, cuff 25 is symmetrically arranged around tubular member 15. Those skilled in the art will appreciate that other arrangements of the cuff are possible, for example, the cuff may be asymmetrically arranged around the elongate tubular member. Cuff 25 is attached to elongate tubular member 15 by any method known to one skilled in the art, for example, by adhesive or heat seal. When attached, cuff 25 will retain material used to inflate it.

An endotracheal tube of the invention may also comprise one or more inflation tubes 30. Typically, an endotracheal tube of the invention will comprise an inflation tube for each cuff with which it is equipped. The inflation tube 30 may be formed as integral part of elongate tubular member 15 or may be a separate tube. When separate, inflation tube 30 may run inside of passage 20. The proximal end of inflation tube 30 remains outside of the patient after installation of endotracheal tube 10 while the distal end of inflation tube 30 is inside of cuff 25. Thus, inflation tube 30 provides fluid communication from the outside of the patient to the interior of cuff 25. The proximal end of inflation tube 30 can be equipped with valve 35. Valve 35 can be used to control the flow of material into and out of cuff 25. In a simple embodiment, valve 35 may consist of a Luer fitting and the fitting may be attached to a syringe of material to be used to inflate cuff 25. Valve 35 may be any other suitable connector for controlling the flow of material from a dispenser through inflation tube 30 and into cuff 25.

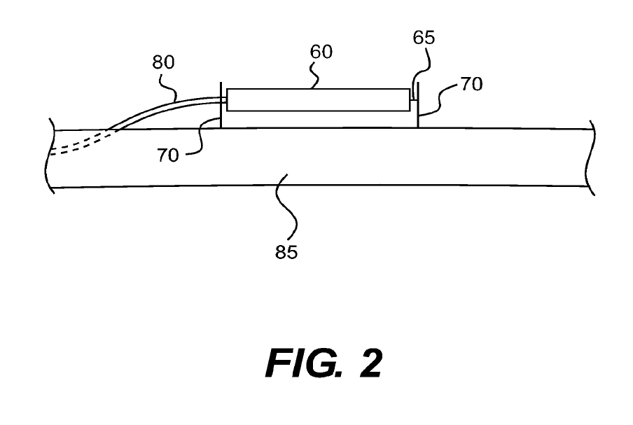

In some embodiments, one or more transducers (e.g., ultrasound transducers, sensors, transmitters etc) may be mounted on endotracheal tube 10 of the invention such that that are adjustable in relationship to the endotracheal tube. Transducers may be adjustable along the axis of the tube, for example, may be moveable along the length of the tube. Transducers may also be tilted laterally in relation to the tube. For example, as shown in FIG. 1, transducer 40, which may be an ultrasound transducer, may be mounted on elongate member 15 such that the direction of the ultrasound emitted by sensor 40 can be varied as needed. For example, with reference to FIG. 2, the transducer 60 may be supported in a bracket 70 mounted to tube 85. A rod or wire 80 may be attached to transducer 60, for example, at the midline of the transducer. The transducer 60 may be equipped with a pin 65 that rotatably engages bracket 70. Rod 80 may be rotated by the user causing transducer 60 to tilt. Adjustably mounted transducers and/or sensors may be controlled using any techniques know in the art, for example, screw drives, gear drives, belt drives, telescoping rods, and/or inflatable bladders.

In some embodiments, sensors may comprise a plurality of ultrasound transducers, e.g., may be an array of ultrasound transducers. For example, transducer 40 may be an array of ultrasound transducers. Any arrangement of transducers known in the art may used in the practice of the invention. Examples of suitable arrays of ultrasound transducers include, but are not limited to, linear arrays, radial arrays, and curvilinear arrays. Such arrays may be used to create images of structures within the body of the patient, for example, images of the heart.

The endotracheal tubes of the invention may be used in any situation in which it is desirable to ensure that the patient airway is maintained. In some embodiments, the endotracheal tubes of the invention may be used in the practice of surgery where a patient is to ventilated and/or treated with anesthesia. In some embodiments, the endotracheal tubes of the invention may be used in conjunction with a cryosurgical procedure. The endotracheal tube of the invention is particularly well suited for monitoring patients in critical care situations.

The present invention has been illustrated and described in detail above. The embodiments described herein should be considered illustrative and not limiting of the scope of the invention. Those of skill in the art will appreciate that various changes and modifications can be made to the embodiments described above and those variations and modification are intended to be within the scope of the invention as set out in the appended claims. All references, patents and other printed materials mentioned above are specifically incorporated herein by reference in their entirety.

What is claimed is:

1. An endotracheal tube, comprising:
an elongate tubular member having a proximal end and a distal end;
a flexible cuff having an interior space positioned on the outer surface of the tubular member; and
at least one sensor positioned in the interior space of the cuff.

2. The endotracheal tube according to claim 1, comprising two or more sensors.

3. The endotracheal tube according to claim 1 comprising 3 or more sensors.

4. The endotracheal tube according to any one of claims 1-3, wherein at least one sensor comprises an ultrasound transducer.

5. The endotracheal tube according to claim 4, wherein the sensor comprising an ultrasound transducer is adapted to detect blood flow.

6. The endotracheal according to claim 5, wherein the cuff contains fluid.

7. The endotracheal tube according to claim 1, wherein at least one sensor is detachable from the elongate tubular member.

8. The endotracheal tube according to claim 1, wherein at least one sensor is movably attached to the elongate tubular member.

9. The endotracheal tube according to claim 1, wherein the interior space of the cuff is filled with liquid and the sensor in the interior space of the cuff comprises an ultrasound transducer.

10. A method of performing a surgical procedure, comprising:
inserting an endotracheal according to claim 1;
using at least one sensor to detect at least one parameter; and
performing the procedure.

11. The method according to claim 10, wherein the procedure comprises applying a cryogen to a target tissue or using the cryogen to create an isotherm in proximity to the tissue.

12. The method according to claim 11, wherein the cryogen is applied to a tissue selected from the group consisting of esophageal tissue, lung tissue, tracheal tissue, laryngeal tissue, pharyngeal tissue and gastric tissue.

13. The method of claim 11, wherein the cryogen comprises a liquefied gas.

14. The method according to claim 11, wherein the cryogen is selected from the group consisting of nitrogen, oxygen, argon, carbon dioxide, nitrogen dioxide, and air.

15. The method according to claim 13, wherein the cryogen is sprayed.

16. The method according to claim 15, wherein the cryogen is sprayed using a catheter.

17. The method according to claim 10, wherein the parameter detected is cardiac output.
